(19) United States
(12) Patent Application Publication
(10) Pub. No.: US 2002/0127551 A1
(43) Pub. Date: Sep. 12, 2002

(54) COMPOSITION COMPRISING RANDOM MIXTURES OF OLIGONUCLEOTIDES

(76) Inventor: ALISON HOPKINS, MID GLAMORGAN (GB)

Correspondence Address: MARSHALL O'TOOLE GERSTEIN, MURRAY & BORUN, 6300 SEARS TOWER, 233 SOUTH WACKER DRIVE, CHICAGO, IL, 60606-6402, US

(21) Appl. No.: 09/485,245

(22) PCT Filed: Aug. 21, 1998
(86) PCT No.: PCT/GB98/02550

(30) Foreign Application Priority Data
Aug. 22, 1997 (GB) 9717972.5

(51) Int. Cl. C12Q 1/68

(52) U.S. Cl. 435/6

(57) ABSTRACT

A labelling composition comprises a random mixture of oligonucleotides which are 6-mers to 8-mers said composition present in a dry state. A method of making labelled probes for a nucleic acid template comprises incubating the template under chain extension conditions with the labelling composition. The use of 6-mers to 8-mers reduces self-annealing, which is a problem with 9-mers in a dried state.

This invention concerns compositions comprising random mixtures of oligonucleotides and their use for labelling nucleic acids by a random prime method.

Feinberg and Vogelstein (1, 2) introduced the use of random sequence hexanucleotides to prime DNA synthesis on denatured template DNA at numerous sites along its length. The primer-template complex is a substrate for the “Klenow” fragment of DNA polymerase 1. By replacing a non-radioactive nucleotide with the radiolabelled equivalent in the reaction mixture, newly synthesised DNA is made radioactive.

Very small amounts of input DNA can be labelled, enabling very high specific activity probes to be produced with relatively small quantities of added nucleotides. These radioactive labelled fragments can then be used as sensitive hybridisation probes for a wide range of filter based applications (3-6).

There are several labelling kits that are commercially available for the labelling of DNA by the random prime method. These include the Multiprime, Megaprime, Rediprime and Fluorescein Gene Images kits available from Amersham International pic. Ready-To-Go kits are available from Pharmacia and High Prime kits are available from Boehringer.

The Multiprime kit was introduced in the 1980s. It provides different tubes containing the different solutions that enable the user to make up labelling mixtures. One such tube contains a random mixture of 6-mer oligonucleotides, another the polymerase enzyme, and another the supply of nucleotides in the reaction buffer. All these separate solutions are stored frozen at −20° C. The purchaser thaws the different solutions, and adds precise quantities of each to the sample of denatured DNA that is to be labelled, including a labelled nucleotide. This reaction is then usually incubated at 37° C. at which temperature, oligonucleotide annealing and chain extension can occur. However, the reaction may also be incubated at lower temperatures such as an ambient room temperature of about 20° C.

The Megaprime kit was introduced commercially in the early 1990s. It is similar to the Multiprime kit, except that 9-mer oligonucleotides are used in place of 6-mers. The Megaprime kit has an advantage over the Multiprime kit, in that 9-mer oligonucleotides anneal more strongly (than do 6-mers) to a DNA target and form a hybrid having a higher melting temperature. Thus 9-mers achieve better and more rapid priming of a target then do 6-mers.

The Rediprime kit was introduced commercially in 1994. It comprises a mixture of 9-mer oligonucleotides with a polymerase enzyme and a supply of nucleotides. The mixture is supplied in a freeze-dried state. The freeze-dried mixture also contains a dye for easy visualisation. Dried kits for performing nucleic acid manipulation experiments were described by Ortlepp and McKay in EP 298 669 entitled “Performing nucleic acid reactions”. The user reconstitutes the mixture by adding liquid containing the DNA template that is to be labelled, and then liquid containing the labelled nucleotide.

The Ready-To-Go kit was introduced during the 1990s. It is based on a random prime solution containing a random mixture of 9-mer or longer oligonucleotides, which solution is dried by a technique described in EP 383 569. A dye is not present. Like the Rediprime kit, the Ready-To-Go kit can be stored at +4° C. or at ambient temperature. Promotional literature emphasises the speed of labelling, which results from the use of 9-mer oligonucleotides.

The High Prime kit is a wet kit containing a random mixture of oligonucleotides. The kit literature does not indicate what length of random oligonucleotides are used, but in the related document EP 649 909 A2, the use of 6-mer, 9-mer, 12-mer and 15-mer is disclosed. No preferred length of random oligonucleotide is given. The solution is stablised by the use of glycerol and can be stored at between about −20° C. and +4° C.

It can be seen that there has been a trend in commercial kits towards the use of longer oligonucleotides, particularly 9-mers or even longer. Going against this trend, it has been determined by Suganuma, A and Gupta, K C (7) that the use of long random primers, especially 9-mers or longer, reduces the priming efficiency of the random primer reaction. These authors worked on solutions which were used without being dried at any stage. The conclusions of these authors conflict with the findings of the present inventors; which findings are to the effect that, when experiments are done with solutions which are not dried, 9-mers provide more rapid and efficient labelling than do 6-mers, and do not give rise to any problem resulting from self-annealing or self-priming. To the best of applicants' knowledge, the conclusions reported by the authors of (7) have not caused the suppliers of random prime kits to use shorter oligonucleotides.

The present invention is based on the discovery that self-annealing occurs when random 9-mers are used in dried predispensed labelling kits, and that this limits their stability and shelf life. The self-annealing occurs during dispensing and storage when the random 9-mers anneal together to form primer-dimers or primer concatemers. These primer complexes become labelled during the normal labelling reaction, which concomitantly reduces the amount of label that is incorporated into copies of the template that are being synthesised during the reaction. Shorter oligonucleotides are not subject to this problem. The problem is specific to 9-mers (and longer oligonucleotides) used in dried kits.

The invention provides a labelling composition comprising a random mixture of oligonucleotides which are 6-mers to 8-mers, said composition present in a dry state. Preferably the composition also contains at least one of: a polymerase enzyme; a supply of nucleotides for chain extension; a labelled nucleotide; a dye; a stabiliser; and a buffer.

As the experimental data below shows, 5-mer oligonucleotides are too short to be useful in dried kits. As the length of the oligonucleotides increases from 6-mers to 9-mers, there is a concomitant increase in the aforementioned self-priming problem. On the other hand, longer oligonucleotides anneal more rapidly and strongly to templates than do shorter ones. Taking into account both these factors, applicants believe that 6-mer oligonucleotides are more preferable than 7-mers which in turn are more preferable than 8-mers.

The random mixture of oligonucleotides is present in a dry state. Various drying techniques are possible, including that described in EP 383 569, and also freeze-drying or lyophilisation which is preferred.

It is possible to use any DNA polymerase enzyme in the labelling reaction, for example Klenow, exonuclease free klenow, DNA polymerase I, T7 DNA polymerase, Sequenase™, Thermosequenase™, so long as the reaction buffer conditions are suitable for the specific enzyme being used.

All four of the nucleotides are preferably present in the composition, whether labelled or unlabelled, and the relative molar concentrations may be adjusted to improve the efficiency of labelling. Also when a labelled nucleotide is present, the equivalent unlabelled nucleotide may also be present to improve the efficiency of labelling, or to control the specific activity of the DNA that is being produced from the labelling reaction.

These compositions enable a DNA template to be used to produce copies which are labelled radioactively, for example, by using either phosphate labelled with P-32 or S-35, or by using H-3 or C-14 base labelled nucleotides. Alternatively non-radioactive labels may be used, for example, fluorescein, biotin, digoxigenin, rhodamine and cyanine dyes, may be incorporated when, for example, covalently linked to the base moiety of the nucleotide.

Any stabiliser may be present to protect the activity of the enzyme, for example, trehalose, sucrose, BSA, gelatin. A dye may also be present to allow the dried pellet to be visualised, before use, and to assist in determining that mixing is thorough.

The invention also includes a method of making labelled probes for a nucleic acid template, which method comprises incubating the nucleic acid template under chain extension conditions with the labelling composition as herein described. Preferably the template is DNA. The inventor has found that random 6-mers can give fast labelling kinetics (10 minutes labelling time) by being present at high concentration in the reaction mixture. A preferred concentration is 2-10 O.D./ml in the final reaction with about 5 O.D./ml being most preferable. A probe labelled in this manner is suitable for use in a Southern hybridisation.

All the results shown in the examples show labelling with dCTP-³²P, but this is only as a means to show, and quantitate the amount of self-priming that occurred in each reaction. The reactions are able to label DNA with other labels, both radioactive and non-radioactive, as indicated elsewhere in this specification.

### References

1. Feinberg, A P and Vogelstein, B, Anal. Biochem., 132: 6-13 (1983).

2. Feinberg, A P and Vogelstein, B, Addendum Anal. Biochem., 137: 266-267 (1984).

3. Southern, E M, J. Mol. Biol., 98: 503-517 (1975).

4. Thomas, P S, Proc. Nat. Acad. Sci., USA, 77: 5201-5205 (1980).

5. Meinkoth, J and Wahl, G, Anal. Biochem, 138: 267-284 (1984).

6. Grunstein, M and Hogness, D S, Proc. Natl. Acad. Sci, USA, 72: 3961-3965 (1975).

7. Sugunuma, A and Gupta, K C, Analytical Biochemistry, 224: 605-608 (1995).

### EXAMPLE 1. MANUFACTURE OF LYOPHILISED REACTIONS WITH DIFFERENT RANDOM PRIMER LENGTHS

All primers were diluted to 50 O.D./ml in water. The number of enzyme units was the same in each reaction (7 units).

The amount of each component solution is as follows for a 6 ml scale.

| | 5 mer | 6 mer | 7 mer | 8 mer | 9 mer |
|---|---|---|---|---|---|
| | reaction mix | reaction mix | reaction mix | reaction mix | reaction mix |
| Nucleotide buffer | 1.998 ml | 1.998 ml | 1.998 ml | 1.998 ml | 1.998 ml |
| Exo-free Klenow | 1200 units | 1200 units | 1200 units | 1200 units | 1200 units |
| (12 μl) 100 units/μl | | | | | |
| Dilution Buffer | 28 μl | 28 μl | 28 μl | 28 μl | 28 μl |
| 5 mer primer | 1.0 ml | | | | |
| 6 mer primer | | 1.0 ml | | | |
| 7 mer primer | | | 1.0 ml | | |
| 8 mer primer | | | | 1.0 ml | |
| 9 mer primer | | | | | 1.0 ml |
| 20% Trehalose | 1.5 ml | 1.5 ml | 1.5 ml | 1.5 ml | 1.5 ml |
| 0.2 mg/ml Xylene | 0.198 ml | 0.198 ml | 0.198 ml | 0.198 ml | 0.198 ml |
| Cyanol | | | | | |
| PF Water | 1.264 ml | 1.264 ml | 1.264 ml | 1.264 ml | 1.264 ml |
| Total Volume | 6 ml | 6 ml | 6 ml | 6 ml | 6 ml |

Each reaction mix was dispensed into tubes 35 μl aliquots, and were freeze dried.

### Methods:

1. Nucleotide buffer: Labelling buffer from Nick Translation kit (N5000/N5500 Amersham International plc).

2. Dilution buffer: Storage buffer for enzyme dilution.

3. Labelling Method: Tubes of DNA for labelling were made up as follows:

5 μl λ HindIII DNA at 5 ng/μl in TE buffer.

40 μl water.

Placed all tubes in a boiling water bath (95 to 100° C.) for 5 minutes,

placed all tubes on ice for 5 minutes, centrifuged briefly,

then added the denatured DNA solutions to the respective dried reaction tube samples

added 5 μl Redivue™ dCTP (α³²P) (Product Code AA0005: Amersham International plc) (50 μl total reaction volume).

Incubated all reactions for 10 minutes at 37° C.

Spotted 2 μl samples out onto PEI-cellulose tic plates,

Ran plates in 1.25 M KH₂PO₄ pH 3.4.

Analysed plates on plate scanner, to measure the %incorporation, %self-priming and %dCTP present at the end of each reaction.

The %self-priming is defined as the % of the total radioactive counts that are situated between the incorporated counts and the counts due to the unincorporated dCTP-³²P.

λ HindIII DNA Labelling with dCTP-³²P (Week 1 Test)

| | | Tube-1 | | | Tube-2 | | |
|---|---|---|---|---|---|---|---|
| | Primer | % | % Self- | % | % | % Self- | % |
| Tube | Type | Incorp | Prime | dCTP | Incorp | Prime | dCTP |
| 1, 2 | 5 mers | 62.7 | 7.9 | 23.0 | 54.8 | 7.7 | 30.9 |
| 3, 4 | 6 mers | 79.9 | 11.2 | 2.7 | 82.1 | 10.8 | 2.2 |
| 5, 6 | 7 mers | 73.5 | 17.5 | 2.8 | 74.3 | 15.1 | 3.7 |
| 7, 8 | 8 mers | 68.3 | 19.1 | 3.2 | 65.6 | 20.4 | 3.6 |
| 9, 10 | 9 mers | 64.9 | 23.7 | 3.1 | 61.5 | 27.2 | 3.1 |

The column headed “% Incorp” shows the percentage of dCTP-³²P incorporated as a chain extension product of a primer-λHind Ill DNA hybrid. The column headed “% Self-Prime” shows the percentage of dCTP-³²P incorporated in a complex involving only primers. The column headed “% dCTP” shows the percent of unincorporated dCTP-³²P. The % dCTP figures were unacceptably high when 5-mer oligonucleotides were used, but were acceptable for 6-mers to 9-mers. Within this range, the % Incorp figures decrease as the oligonucleotide length increases from 6 to 9.

### EXAMPLE 2. LONG TERM STABILITY COMPARISON OF DRIED REACTIONS 15 nonamers compared with hexamers, 3.5 units of Exo-free Kienow per reaction:

The samples were made up as shown in Example 1, but 6 μl of Exo-free Klenow was used.

DNA Labelling with dCTP-³²P, results are the averages of the three
reactions

| | Nonamers | | | Hexamers | | |
|---|---|---|---|---|---|---|
| | | % Self- | | | % Self- | |
| Week | % Incorp | Prime | % dCTP | % Incorp | Prime | % dCTP |
| 3 | 61.9 | 17.5 | 6.2 | 69.6 | 9.7 | 6.3 |
| 6 | 71.4 | 18.0 | 4.3 | 80.8 | 8.4 | 4.7 |
| 10 | 65.8 | 20.2 | 6.4 | 75.0 | 11.9 | 6.9 |
| 16 | 66.5 | 16.5 | 8.0 | 73.4 | 11.1 | 6.5 |
| 21 | 78.3 | 10.8 | 3.0 | 84.3 | 5.8 | 2.4 |
| 25 | 42.7 | 11.6 | 40.4 | 55.1 | 5.4 | 35.1 |

As these figures show, the % incorporation of dCTP-³²P when using 9-mers was initially lower than when using 6-mers and remained lower storage of the compositions for up to 25 weeks.

### EXAMPLE 3:

Using dried reactions as shown in Example 1, the primer was replaced with water for the reaction drying, and was added later as a separate solution, when the reactions were being used. All reactions were incubated for 10 minutes, and then sampled to measure the % incorporation.

| Primer Concentration | % Incorporation | % Incorporation |
|---|---|---|
| in reaction | (hexamer primers) | (nonamer primers) |
| O.D./ml | Average of two reactions | one reaction |
| 6.0 | 78.3 | |
| 5.0 | 83.2 | 81.0 |
| 4.0 | 67.7 | 65.6 |
| 2.0 | 51.5 | 67.0 |
| 1.0 | 45.1 | 60.2 |

It can be seen from these results that the same primer concentration (O.D./ml) is required to achieve the same reaction kinetics, i.e. the same % incorporation in 10 minutes with different random primer lengths. This shows that the molar concentration needs to increase as the primer length is reduced.

Although the above results were obtained using wet reagents, the conclusion would apply also when dry primers are used.

EXAMPLE 4:

Densitometer results of Southern hybridisations

25ng labelling reactions were carried out using the Megaprime Labelling Kit RPN 1606 (Amersham International plc) or using labelled probes from dried nonamer or hexamer labelling reactions made as described above in other examples. Southern blots were hybridised for 2 hours at 65° C. with the labelled probe under standard conditions and then washed in 2×SSC, 0.1% SDS, 20 minutes at room temperature, followed by two washes in 0.5×SSC, 0.1% SDS, for 5 minutes 65° C. The dried blots were detected on X-ray film with 2 intensifying screens and place into a −700° C. freezer, for 16 hours. After the film was developed using a film processor it was scanned using a densitometer, then the results were analysed using ImageQuant software.

| | Time of test | | % band intensity of |
|---|---|---|---|
| | after | | Southern hybridisation |
| Kit | manufacture | Target | of Megaprime control |
| 9 mers | 1 week | 0.25 pg | 42.23 |
| 9 mers | 1 week | 0.5 pg | 40.12 |
| 9 mers | 1 week | 1.0 pg | 38.93 |
| 6 mers | 1 week | 0.25 pg | 97.09 |
| 6 mers | 1 week | 0.5 pg | 95.02 |
| 6 mers | 1 week | 1.0 pg | 94.33 |
| 6 mers | 37 weeks | 0.25 pg | 74.58 |
| 6 mers | 37 weeks | 0.5 pg | 80.91 |
| 6 mers | 37 weeks | 1.0 pg | 81.17 |

Conclusions:

The hexamers used in a dried labelling reaction generate labelled probes which gave a much stronger band intensity than when nonamers are used, not only when tested initially after 1 week, but even after an extended period of storage (37 weeks at room temperature).

1. A labelling composition comprising a random mixture of oligonucleotides which are 6-mers to 8-mers, said composition present in a dry state.

2. A labelling composition as claimed in claim 1, wherein the composition also contains at least one of: a polymerase enzyme; a supply of nucleotides for chain extension; a labelled nucleotide; a dye; a stabiliser; and a buffer.

3. A labelling composition as claimed in claim 1 or claim 2, wherein the random mixture is of 6-mer oligonucleotides.

4. A labelling composition as claimed in any one of claims 1 to 3, wherein the composition is present in a freeze-dried state.

5. A method of making labelled probes for a nucleic acid template, which method comprises incubating the nucleic acid template under chain extension conditions with the labelling composition of any one of claims 1 to 4.

6. A method as claimed in claim 5, wherein the random mixture of oligonucleotides is present at a concentration of 2-10 O.D./ml.
